SEQUENCE LISTING (18 sequences)

SEQ ID NO: 1 - DNA (genomic), 1028 bp; single-stranded, linear
  CDS 49..795
  sig_peptide 49..100
  mat_peptide 103..795
cacgagatttcatgagcatcctcctctaaacgcgtgtcaagacaaaagatgcttcagctttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaaggcatccttgagaaactgaaggtcgacctaggagtgcttcagaaatccagtgcttggcaactggccaagcagaaggcccaggaagctgagaaattgctgaacaatgtcatttctaagctgcttccaactaacacggacatttttgggttgaaaatcagcaactccctcatcctggatgtcaaagctgaaccgatcgatgatggcaaaggccttaacctgagcttccctgtcaccgcgaatgtcactgtggccgggcccatcattggccagattatcaacctgaaagcctccttggacctcctgaccgcagtcacaattgaaactgatccccagacacaccagcctgttgccgtcctgggagaatgcgccagtgacccaaccagcatctcactttccttgctggacaaacacagccaaatcatcaacaagttcgtgaatagcgtgatcaacacgctgaaaagcactgtatcctccctgctgcagaaggagatatgtccactgatccgcatcttcatccactccctggatgtgaatgtcattcagcaggtcgtcgataatcctcagcacaaaacccagctgcaaaccctcatttgaagaggacgaatgaggaggaccactgtggtgcatgctgattggttcccagtggcttgccccacccccttatagcatctccctccaggaagctgctgccaccacctaaccagcgtgaaagcctgagtcccaccagaaggaccttcccagataccccttctcctcacagtcagaacagcagcctctacacatgttgtcctgcccctggcaataaaggcccatttctgcaaaaa

SEQ ID NO: 2 - protein, 249 aa
MLQLWKLVLLCGVLTGTSESLLDNLGNDLSNVVDKLEPVLHEGLETVDNTLKGILEKLKVDLGVLQKSSAWQLAKQKAQEAEKLLNNVISKLLPTNTDIFGLKISNSLILDVKAEPIDDGKGLNLSFPVTANVTVAGPIIGQIINLKASLDLLTAVTIETDPQTHQPVAVLGECASDPTSISLSLLDKHSQIINKFVNSVINTLKSTVSSLLQKEICPLIRIFIHSLDVNVIQQVVDNPQHKTQLQTLI

SEQ ID NO: 3 - protein, 235 aa
MFQLGSLVVLCGLLIGNSESLLGELGSAVNNLKILNPPSEAVPQNLNLDVELLQQATSWPLAKNSILETLNTADLGNLKSFTSLNGLLLKINNLKVLDFQAKLSSNGNGIDLTVPLAGEASLVLPFIGKTVDISVSLDLINSLSIKTNAQTGLPEVTIGKCSSNTDKISISLLGRRLPIINSILDGVSTLLTSTLSTVLQNFLCPLLQYVLSTLNPSVLQGLLSNLLAGQVQLAL

SEQ ID NO: 4 - protein, 235 aa
MFQLGSLVVLCGLLIGTSESLLGDVANAVNNLDILNSPSEAVAQNLNLDVGSLQQATTWPSAKDSILETLNKVELGNSNGFTPLNGLLLRVNKFRVLDLQAGLSSNGKDIDLKLPLVFEISFSLPVIGPTLDVAVSLDLLNSVSVQTNAQTGLPGVTLGKCSGNTDKISISLLGRRLPFVNRILDGVSGLLTGAVSILLQNILCPVLQYLLSTMSGSAIQGLLSNVLTGQLAVPL

SEQ ID NO: 5 - protein, 206 aa
MFQLGSLVVLCGLLIGTSGSLFDIFQNPELDVESVWSEINYRIRYALETMDLDMLADYLSKRGIELKIKDLRILNLNHEVSPNGDEVTLKMPMALNASLSLPARDLTTDVSISMEAITSFAIEKDPKTGRRVLNMQRCSLNTDNTSISLLNRKSNFVNLALDSALYLIKRGLTLPVRRQLCPVLQLIISNTFHPDEISNPQTAIST

SEQ ID NO: 6 - DNA (genomic), 39 bp; single-stranded, linear
ctacagccatggagtctcttcttgacaatcttggcaatg

SEQ ID NO: 7 - DNA (genomic), 36 bp; single-stranded, linear
catcgcggatccaatgagggtttgcagctgggtttt

SEQ ID NO: 8 - DNA (genomic), 43 bp; single-stranded, linear
ctacgcggatccgccatcatgcttcagctttggaaacttgttc

SEQ ID NO: 9 - DNA (genomic), 31 bp; single-stranded, linear
ctctgctctagactaaatgagggtttgcagc

SEQ ID NO: 10 - DNA (genomic), 449 bp; single-stranded, linear
ggcacgagatttcatgagcatcctcctctaaacgcgtgtcaagacaaaagatgcttcagctttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaagggcatccccnttttngagaaactgaaggtcgacctaggagtgcttcagaaatccagtgcttggcaactggccaagcagaaggcccaggaagctgagaaattgctgaaccaatgtcatttctaagctgcttccaactaacacggacatttttggggtgaaaaatcagcaactccctcatcctggatgtcaaagctgaaccgatcgatgatggnaaaggcttaaactggagcttccctgtcanc

SEQ ID NO: 11 - DNA (genomic), 538 bp; single-stranded, linear
atgagcatcctcctctaaacgcgtgtcaagacaaaagatgctnncagctttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaaggcatccttgagaaactgaaggtcgacctaggagtgcttcagaaatccagtgcttggcaactggccaacagaaggnccaggaagctgagaaattgctgaacaatgtcatttctaagctgcttccaactaacacggacatttttgggttgaaantcagcaatncccncanccggatgttcaaagntgnancgatcgatgatgggcaaaggctttaanccggaggcttccctgttcaccgggaatgttcaangtnggcccgggcccntcattgggccagnttatcaaanctggaaagcttcctgggacctccggacnggntcaacaattgaaangattccccgana

SEQ ID NO: 12 - DNA (genomic), 359 bp; single-stranded, linear
ggcacgagatttcatgagcatcctcctctaaacgcgtgtcaagacaaaagatgcttcagctttggaaacttgttctcctgtgcggcgtgctcactgggacctcagagtctcttcttgacaatcttggcaatgacctaagcaatgtcgtggataagctgggaacctgttcttcacgagggacttgagacagttgacaatactcttaaaggcatccttgagaaactgaaggtcganctaggagtgcttcagaaatccagtgcttggcaactggncaagcagaaggncccaggaagctgagaaattgctggancaatgtcaattctaagctgnttccgactaacacggncatttttgggttg

SEQ ID NO: 13 - DNA (genomic), 374 bp; single-stranded, linear
acccacgcgtccggcgtgatcaacacgctgaaaagcactgtatcctccctgctgcagaaggagatatgtccactgatccgcatcttcatccactccctggatgtgaatgtcattcagcaggtcgtcgataatcctcagcacaaaacccagctgcaaaccctcatctgaagaggacgaatgaggaggaccactgtggtgcatgctgattggttcccagtggcttgccccacccccttatagcatctccctccaggaagctgctgccaccacctaaccagcgtgaaagcctggagtcccaccagaaggaccttcccagatacccctttttcctcacagtcagaggnggnngcctcttacacntgttgtccnggccc

SEQ ID NO: 14 - DNA (genomic), 406 bp; single-stranded, linear
ttcatgagcatcctcctctaaacgcgtgtcaagacaaaagatgcttcactttggaaacttgttctcctgtgcggcgtgctcactgggacctcagagtctcttcttgacaatcttggcaatgacctaagcaatgtcgtggataagctggaacctgttcttcacgagggacttgagacagttgacaatactcttaaaggcatccttgagaaactgaaggtcgacctaggagtgcttcagaaatccagtgcttggcaactgggccancagaaaggcccagggaaagcggagaaattgctggaacaatgttcatttctaaagctgctttccaactaacacgggacntttttggggtttgnaaaatcagccaacttccctcaaccnnggatgttccaaagctggaaaccgn

SEQ ID NO: 15 - DNA (genomic), 493 bp; single-stranded, linear
tcgacccacgcgtccgccgcccccaactttttggcagttctcccatctcttgcccacttgaccagactttaatagttccctgtgttttccagacacagccaaatcatcaacaagttcgtgaatagcgtgatcaacacgctgaaaagcactgtatcctccctgctgcagaaggagatatgtccactgatccgcatcttcatccactccctggatgtgaatgtcattcagcaggtcgtcgataatcctcagcacaaaacccagctgcaaaccctcatctgaagaggacgaatgaggaggaccactgtggtgcatgctggtgaggagccagtctctgtgccccaatgcacaggggcctatggtgaagtaaaagtcaagcgtggcttcccttatttttgtgttagaagactgtgccttcatctcagtcatagattgagccctggnccccatcccangctaaggcctgattctggtcanactctgaacactgagcctt

SEQ ID NO: 16 - DNA (genomic), 395 bp; single-stranded, linear
tcgacccacacgtccgcctgactgacagaaggagatatgtccatgatccgacattcttacatccactccctggatgtgaatgttcattncagcaggtcgtncgataatccngcagcacaaaacccagctggcaaaccctncatctgaagangacgaatgaggangaccactgtgggtgcatgctgattggtntcccagtgggcttgccccaacccccttanagcanctccctccaggaagctgctgcnaaccaaccgaaccagcgtgaaagcctgaatnccaccagaaggaccttcccagatanccctgctncncaacagtnaagaacagcagcttcgaacaacatgnggttctggcccccgggcaataaaaggcccattttggcaaaaaaaaaa

SEQ ID NO: 17 - DNA (genomic), 116 bp; single-stranded, linear
ggcacganatttcatgagcatcctcctctaaacacgtgtcaagacaaaagatgcttcagctttggnaacttgttctcctatncngcgtgctcactgngacctcagaatctctnctt

SEQ ID NO: 18 - DNA (genomic), 360 bp; single-stranded, linear
gtggnccggggcccatcattgggccaganttatcaacctgaaagcctccnngganctcctgaccgcagtcaacaattggaaactggatcccccagaacaacaaccagcctggttgnccgtnctggggaagaatgccgnccaatgaancccaaaccaagcancttcactnntncctnggctggggaccaaaacaccaggccaaaatccantnaancaagtntccgtggnaataagcgtggaatccaaacaacgctgggaaaaagcantgggnatnccntccctggctgggcaagaaagggngatatggtccactggaatccggaatttttaanccaattccctnggaatgtggnaatgtca

(19) United States
(12) Patent Application Publication
(10) Pub. No.: US 2002/0132304 A1
(43) Pub. Date: Sep. 19, 2002

(54) Human parotid secretory protein

(75) Inventors: Roxanne Duan, Bethesda, MD (US); Steven M. Ruben, Olney, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD (US)

(21) Appl. No.: 10/020,139

(22) Filed: Dec. 18, 2001

Related U.S. Application Data
(63) Continuation of application No. 08/993,529, filed on Dec. 18, 1997, now abandoned.
(60) Provisional application No. 60/034,429, filed on Dec. 23, 1996.

(51) Int. Cl. C12P 21/02; C12N 5/06; C07H 21/04; C07K 14/575

(52) U.S. Cl. 435/69.4; 435/325; 435/320.1; 536/23.5; 530/399

(57) ABSTRACT

The present invention relates to a novel human parotid secretory protein (hPSP) protein which is a member of the PSP family. In particular, isolated nucleic acid molecules are provided encoding the human hPSP protein. hPSP polypeptides are also provided as are vectors, host cells and recombinant methods for producing the same. The invention further relates to screening methods for identifying agonists and antagonists of hPSP activity. Also provided are diagnostic methods for detecting digestive, nonimmune defense, endocrine or immune system-related disorders and therapeutic methods for treating such disorders.

### FIELD OF THE INVENTION

The present invention relates to a novel human gene encoding a polypeptide which is a member of the Parotid Secretory Protein family. More specifically, isolated nucleic acid molecules are provided encoding a human polypeptide named Human Parotid Secretory Protein, hereinafter referred to as hPSP. hPSP polypeptides are also provided, as are vectors, host cells and recombinant methods for producing the same. Also provided are diagnostic methods for detecting disorders related to the digestive, endocrine and immune systems and therapeutic methods for treating such disorders. The invention further relates to screening methods for identifying agonists and antagonists of hPSP activity.

This application claims benefit of 35 U.S.C. section 119(e) based on copending U.S. Provisional Application Ser. No. 60/034,429, filed Dec. 23, 1996.

### BACKGROUND OF THE INVENTION

Secretion of saliva by major and minor salivary glands, including the main paired sublingual, submaxillary and parotid glands, is critical to the maintenance of all oral tissues. See, for instance, Fox, P. C. et al., “Secretion of Antimicrobial Proteins from the Parotid Glands of Different Aged Healthy Persons,” J. Gerentol. 42:466-469 (1987). Because the oral cavity is exposed to the external environment, a major role of saliva is to offer protection against an almost limitless variety of insults. An especially important salivary function is controlling bacterial colonization of the mouth. Saliva contains many antimicrobial proteins, including bother antibodies and nonimmune defense proteins; and it is believed that the presence of these proteins prevents the common occurrence of oral infections as well as inhibits the systemic access of serious pathogens. For instance, among the known antimicrobial proteins produced in human saliva by the oral exocrine system are the “histatins,” a family of histidine rich proteins with antimicrobial (e.g., anticandidial or antibacterial) activities. See, for example, Xu, T., et al., Infect. Immun. 59:2549-2554 (1991) and Nishikata, M., et al., Biochem. Biophys. Res. Commun. 174:625-630 (1991).

Saliva also is considered a tool for detection of systemic disease, and for monitoring hormones, drugs and pollutants, since saliva can be collected by simple methods and is easily stored and analyzed. See, for instance, Mogi, M., et al, “Analysis and identification of human parotid salivary proteins by micro two-dimensional electrophoresis and Western-blot techniques,” Archs. oral Biol. 31:337-339 (1986). Toward this end, human salivary proteins, including parotid salivary proteins, have been analyzed by electrophoretic and immunological techniques, and a two dimensional “map” of 62 proteins has been prepared, of which 20 were identified as known proteins having antimicrobial or digestive functions. Id. at 339.

Salivary gland secretion is influenced by many clinical situations, including a large number of pharmaceuticals commonly used by older individuals (e.g., antidepressants, antihypertensives, diuretics). Fox et al., supra, 1987. For instance, diabetes, a pathological state reflecting the loss of insulin, has been associated with altered salivary secretion and mouth dryness. Wang, P-L et al., “Effect of chronic insulin administration on mouse parotid and submandibular gland function,” Proc. Soc. Exp. Biol. Med. 205:353-361 (1994). While in diabetic animals insulin increases secretion of some major salivary proteins, particularly amylase, increased insulin concentrations in normal mice reduced salivary concentrations of amylase but increased salivary levels of Epidermal Growth Factor (EGF), and also resulted in hypertrophy and hyperplasia of the parotid and submandibular glands. Similarly, chronic treatment of mice and rats with the beta adrenergic agonist isoproterenol (IPR) causes marked hypertrophy and hyperplasia of the salivary glands and alters the expression level of several secretory proteins. Vugman, I. and A. R. Hand, Microscopy Res. Tech. 31:106-117 (1995). Thus, levels of amylase and a major parotid secretory protein (i.e., protein immunologically cross-reactive with a rat homologue of the main mouse parotid secretory protein (PSP) fell dramatically after IPR treatment, then increased during recovery after cessation of that treatment.

The adult parotid gland is composed mainly of two cell types, acinar and interloblar duct cells. See, for instance, Shaw, P. et al., “Developmental coordination of α-amylase and PSP gene expression during mouse parotid gland differentiation is controlled postranscriptionally,” Cell,47:107-112 (1986). The acinar cells, which represent 75 to 85% of the cells of the tissue, are the site of secretory protein synthesis. The postnatal development of the parotid gland can be roughly divided into two time periods, in the mouse (Mus muscalus), birth to two weeks and two weeks to adult. The first phase is characterized by active acinar cell enlargement and elaboration of the rough endoplasmic reticulum (REP). Two very abundant salivary proteins are produced by these cells, α-amylase (AMY-1, a digestive enzyme) and the major parotid secretory protein (PSP). Id.; see also, Shaw, P. and Schibler, U., “Structure and expression of the Parotid Secretory Protein gene of mouse,” J Mol. Biol. 192:567-576 (1986); Madsen, O., and J. P. Hjorth, “Molecular cloning of mouse PSP mRNA,” Nuc. Acids Res. 13:1-13 (1985). The mRNAs encoded by these two genes accumulate to very high levels in the adult mouse gland, constituting approximately 2% and 10% of the poly(A)⁺ RNA, respectively. Thus, the 1000 nucleotide mRNA encoded by this gene accumulates to approximately 5×10⁴ molecules per parotid acinar cell, thus representing the most abundant mRNA in this tissue.

In the mouse, Shaw & Schilber, 1986, supra, detected mRNA hybridizable to a PSP cDNA only in the parotid gland and not in the other salivary glands nor in the pancreatic gland or any of eleven other tested tissues. However, Poulsen et al. found mRNA hybridizable to a mouse PSP cDNA not only in the parotid gland, but also in considerably smaller amounts in the submaxillary glands, and in even lower amounts in pancreas (Poulsen, K. et al., EMBO J. 5:1891-1896 (1986)). The mouse PSP gene is composed of eight introns and nine exons. The PSP transcription unit measures 8300 bases from cap nucleotide to poly(A) addition site. The structural locus for the PSP gene is located on chromosome 2. Using a DNA construct, named Lama, derived from the mouse PSP gene, salivary gland specific gene expression was obtained in transgenic mice. Mikkelsen, T. R., et al., Nuc. Acids Res. 20:2249-2255 (1992). It was found that 4.6 kb of 5′ flanking sequence is sufficient to direct expression specifically to the salivary glands.

The 22,000 Mᵣ preprotein encoded by the 1,000 nt mouse PSP mRNA is cleaved to yield a 20,000 Mr protein found in the saliva of mice. Analysis of PSP and amylase protein levels in a wide variety of mouse strains indicates a constant PSP to amylase ratio of about five over a large variation in absolute levels of synthesis, suggesting co-ordinate regulation of these two genes.

Cloned mouse PSP cDNAs have been shown to hybridize to mRNAs in parotid glands of rat, white-faced fieldmouse, and bank vole (Poulsen, K., et al., supra). Mouse PSP cDNA also has been shown to hybridize in Southern blots to human leukocyte DNA preparations (Madsen & Hjorth, 1985, supra at page 11), and to mRNA present in both human parotid and submandibular glands (Id.) Rat cDNAs homologous to the mouse PSP gene have been isolated (Madsen & Hjorth, 1985, supra), and a rat gene homologous to mouse PSP also has been called “PS-5” by Shaw, P. et al. (Gene 29:77-85, 1984).

More recently, evidence has been presented that rat PSP and a homologous neonatal rat submandibular gland protein (“SMG-A”) are alternatively regulated members of a salivary protein multigene family. Mirels, L.. and W. D. Ball, J. Biol. Chem. 267:2679-2687 (1992). The acinar cells, which synthesize and secrete salivary proteins, are characterized as serous or mucous by morphologic criteria and by their ability to produce salivary mucin glycoproteins. In the rat, the serous cells of the major salivary glands are the parotid acinar and sublingual serous demilune cells. The acinar cells of rat sublingual and submandibular glands are mucin-producing. Each secretory cell type synthesizes a unique complement of salivary proteins. On the basis of salivary proteins that have been characterized to date, there appears to be some overlap in the proteins produced by the serous or mucous cells of different salivary glands, but little similarity between the products of serous and mucous cells.

Submandibular gland-A (SMG-A) protein is a major secretory product of the neonatal rat submandibular gland but is not synthesized by the acinar cells of the adult gland. Id. The leucine-rich protein is a predominant product of the adult rat parotid gland. cDNA clones encoding SMG-A and the leucine-rich protein were identified by homology to mouse PSP and characterized. The leucine-rich protein shares extensive sequence homology with mouse PSP throughout its 5′- untranslated, protein coding and 3′ untranslated regions, prompting the suggestion that the leucine-rich protein should be referred to as rat PSP. SMG-A is more divergent, having greatest identity (i.e., about 30% amino acid identity) with rat and mouse PSP in its signal peptide and 3′ untranslated sequences. Transcripts homologous to SMG-A and rat PSP, but more closely related to SMG-A, were also identified in rat sublingual gland and mouse sublingual and lacrimal gland by Northern blot analysis. Accordingly, rat SMG-A and PSP appear to arise from alternatively regulated members of a multigene family also including one or more sublingual gland homolog(s).

Alignment of the SMG-A, mouse PSP and rat PSP amino acid sequences reveals that these proteins share one notable region of identity in addition to their signal peptides. Id. The secreted forms of all three proteins contain two conserved Cys residues (mouse and rat PSP residues 161 and 204, SMG-A 138 and 181) separated by an identical distance. The amino acids clustered around these residues are notably more identical that the remainder of the secreted proteins. Therefore, it has been suggested that this relatively conserved region may be functionally important to PSP and SMG-A and may contribute to immunologic cross-reactivity observed (Ball, W. D., et al., Critical Rev. Oral Biol. and Med. 4:517-524 (1993)) between these two proteins.

The members of the PSP family share certain regulatory features. Id. First, PSP, SMG-A and their sublingual gland (SLG) homolog have been immunolocalized to the parotid acinar, submandibular type III, and sublingual serous demilune cells, all of which are serous. The lacrimal gland is also serous and morphologically similar to the salivary glands. No immunoreactive protein as been detected in the mucous acinar cells of the rat sublingual or adult submandibular gland. The PSP family members are also all abundant transcripts of their corresponding cell type. Finally, another regulatory feature which appears to be common to PSP family members is that of being among the earliest secretory products of the developing salivary glands. Thus, the initiation of murine PSP transcription occurs at higher levels earlier in development than that of amylase. (Shaw et al., 1986, supra; Poulsen et al., 1986, supra, cited in Mirels & Ball, 1992, supra), and transcription of the SMG-A and sublingual homolog have also been show to rise dramatically between 18 and 20 days of gestation. Accordingly, the PSP gene family has diverged to include several salivary gland-specific members which retain the common traits of early and abundant expression.

Isolation and characterization of a rat nontumorigenic parotid acinar cell clone, human nontumorigenic parotid acinar cell clone, and a human tumorigenic acinar clone have been reported recently. Prasad, K. N., et al., In Vitro Cell. Dev. Biol. -Animal 31:767-772 (1995). The authors particularly noted that the level of PSP, measured with both immunological and nucleic acid hybridization methods with mouse PSP reagents, increased upon transformation of human nontumorigenic acinar cells to cancer cells, although in vivo levels of PSP in human parotid glands were higher than in either tumorigenic or nontumorigenic human acinar clones.

Thus, there is a need for human polypeptides that function in saliva and elsewhere in the regulation of digestive functions and nonimmune defense mechanisms which are protective against infections, since disturbances of such regulation may be involved in digestive system disorders and disorders relating to infections caused by ingested food or materials. Therefore, there is a need for identification and characterization of such human polypeptides and genes encoding them, which can play a role in detecting, preventing, ameliorating or correcting such disorders as well identifying the cell and tissue types in which they are expressed, including tumorigenic cell types.

### SUMMARY OF THE INVENTION

The present invention provides isolated nucleic acid molecules comprising a polynucleotide encoding at least a portion of the human Parotid Secretory Protein (hPSP) polypeptide having the complete amino acid sequence shown in FIG. 1 (SEQ ID NO:2) or the complete amino acid sequence encoded by the cDNA clone deposited in a bacterial host as ATCC Deposit Number 97811 on Nov. 26, 1996. The nucleotide sequence determined by sequencing the deposited hPSP clone, which is shown in FIG. 1 (SEQ ID NO: 1), contains an open reading frame encoding a complete polypeptide of 249 amino acid residues, including an initiation codon encoding an N-terminal methionine at nucleotide positions 49-51, and a predicted molecular weight of about 27 kDa. The encoded polypeptide has a predicted leader sequence of 18 amino acids underlined in FIG. 1 (amino acids −18 to −1 in SEQ ID NO:2); and the amino acid sequence of the predicted mature hPSP protein is also shown in FIG. 1, and as amino acid residues 1-231 in SEQ ID NO:2. The hPSP amino acid sequence (SEQ ID NO:2) shares extensive sequence homology with three known murine members of the salivary gland secretory protein multigene family (Mirel & Ball, 1992, supra), including the mouse and rat PSP as well as the rat submaxillary gland-A (SMG-A.) proteins. Nucleic acid molecules of the invention include those encoding the complete amino acid sequence excepting the N-terminal methionine shown in SEQ ID NO: 2, or the complete amino acid sequence excepting the N-terminal methionine encoded by the cDNA clone in ATCC Deposit Number 97811, which molecules also can encode additional amino acids fused to the N-terminus of the hPSP amino acid sequence.

The deposited cDNA clone was discovered in a cDNA library derived from human salivary gland tissue. Northern blot analyses of mRNAs from various other human tissues showed expression of hPSP-related mRNA only in the salivary gland, however, a weak signal was also seen in the pancreas and thymus. Extensive searching for homologous cDNA clones in a database of nucleotide sequences in a wide variety of human cDNA libraries from many different tissues failed to find any cDNA clones identical to any portion (e.g., any contiguous 30 nt) of the hPSP sequence in FIG. 1 (SEQ ID NO: 1), indicating most likely that the abundance of the hPSP mRNA in salivary gland tissue is substantially greater than in pancreas or thymus. It is believed that the weak signal observed in pancreas and thymus is due to cross-hybridization with a message encoding a related protein. Therefore, polynucleotides and polypeptides comprising all or a portion of the hPSP sequences of the invention provides, among other utilities, tissue-specific markers for human salivary gland tissue in particular, as well as thymic and pancreatic tissue, which can be used, for instance, in identifying the source organ of a tissue specimen (either normal or cancerous). In addition, hPSP polypeptides can be used (e.g., in pharmaceutical compositions) to provide antimicrobial (antifungal, antibacterial, antiparasite and antiviral) activities and digestive activities associated with hPSP polypeptides produced in normal human saliva.

Thus, one aspect of the invention provides an isolated nucleic acid molecule comprising a polynucleotide comprising a nucleotide sequence selected from the group consisting of: (a) a nucleotide sequence encoding the hPSP polypeptide having the complete amino acid sequence in SEQ ID NO: 2; (b) a nucleotide sequence encoding the hPSP polypeptide having the complete amino acid sequence in SEQ ID NO: 2 excepting the N-terminal methionine (i.e., positions −17 to 231 of SEQ ID NO: 2); (c) a nucleotide sequence encoding the predicted mature hPSP polypeptide having the amino acid sequence at positions 1 to 231 in SEQ ID NO: 2; (d) a nucleotide sequence encoding the hPSP polypeptide having the complete amino acid sequence excepting the N-terminal methionine encoded by the cDNA clone contained in ATCC Deposit No. 97811 (e) a nucleotide sequence encoding the hPSP polypeptide having the complete amino acid sequence encoded by the cDNA clone contained in ATCC Deposit No. 97811; (f) a nucleotide sequence encoding the mature hPSP polypeptide having the amino acid sequence encoded by the cDNA clone contained in ATCC Deposit No. 97811; and (g) a nucleotide sequence complementary to any of the nucleotide sequences in (a), (b), (c), (d), (e) or (f) above.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e), (f) or (g), above, or a polynucleotide which hybridizes under stringent hybridization conditions to a polynucleotide in (a), (b), (c), (d), (e), (f) or (g), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a hPSP polypeptide having an amino acid sequence in (a), (b), (c), (d), (e) or (f), above.

The present invention also relates to recombinant vectors, which include the isolated nucleic acid molecules of the present invention, and to host cells containing the recombinant vectors, as well as to methods of making such vectors and host cells and for using them for production of hPSP polypeptides or peptides by recombinant techniques.

The invention further provides an isolated hPSP polypeptide comprising an amino acid sequence selected from the group consisting of: (a) the amino acid sequence of the full-length hPSP polypeptide having the complete amino acid sequence shown in SEQ ID NO: 2 or the complete amino acid sequence encoded by the cDNA clone contained in the ATCC Deposit No. 97811 (b) the amino acid sequence of the full-length hPSP polypeptide having the complete amino acid sequence shown in SEQ ID NO: 2 excepting the N-terminal methionine (i.e., positions −17 to 231 of SEQ ID NO: 2) or the complete amino acid sequence excepting the N-terminal methionine encoded by the cDNA clone contained in the ATCC Deposit No. 97811; (c) the amino acid sequence of the mature hPSP shown in SEQ ID NO: 2 at positions 1 to 231 or the mature hPSP polypeptide having the amino acid sequence encoded by the cDNA clone contained in the ATCC Deposit No. 97811. The polypeptides of the present invention also include polypeptides having an amino acid sequence at least 80% identical, more preferably at least 90% identical, and still more preferably 95%, 96%, 97%, 98% or 99% identical to those described in (a), (b) or (c) above, as well as polypeptides having an amino acid sequence with at least 90% similarity, and more preferably at least 95% similarity, to those above.

An additional embodiment of this aspect of the invention relates to a peptide or polypeptide which comprises the amino acid sequence of an epitope-bearing portion of an hPSP polypeptide having an amino acid sequence described in (a), (b) or (c), above. Peptides or polypeptides having the amino acid sequence of an epitope-bearing portion of an hPSP polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

In another embodiment, the invention provides an isolated antibody that binds specifically to an hPSP polypeptide having an amino acid sequence described in (a), (b) or (c) above. The invention further provides methods for isolating antibodies that bind specifically to an hPSP polypeptide having an amino acid sequence as described herein. Such antibodies are useful diagnostically or therapeutically as described below.

The invention further provides compositions, including pharmaceutical compostions, comprising an hPSP polynucleotide or an hPSP polypeptide for administration to cells in vitro, to cells ex vivo and to cells in vivo, or to a multicellular organism. In certain particularly preferred embodiments of this aspect of the invention, the compositions comprise an hPSP polynucleotide for expression of an hPSP polypeptide in a host organism for treatment of disease. Particularly preferred in this regard is expression in a human patient for treatment of a dysfunction associated with aberrant endogenous activity of hPSP

In another aspect, a screening assay for agonists and antagonists is provided which involves determining the effect a candidate compound has on hPSP binding to an hPSP binding molecule such as an antibody or receptor. In particular, the method involves contacting the hPSP binding molecule with an hPSP polypeptide and a candidate compound and determining whether hPSP polypeptide binding to hPSP binding molecule is increased or decreased due to the presence of the candidate compound. In this assay, an increase in binding of hPSP over the standard binding indicates that the candidate compound is an agonist of hPSP binding activity and a decrease in hPSP binding compared to the standard indicates that the compound is an antagonist of hPSP binding activity.

It has been discovered that mRNA related to the hPSP gene is expressed not only in salivary gland tissue but also in the pancreas and thymus. For a number of disorders of systems involving these tissues or cells, particularly of the digestive, nonimmune defense, endocrine, and immune systems, significantly higher or lower levels of hPSP gene expression may be detected in certain tissues (e.g., cancerous and wounded tissues) or bodily fluids (e.g., particularly saliva, but also serum, plasma, urine, synovial fluid or spinal fluid) taken from an individual having such a disorder, relative to a “standard” hPSP gene expression level, i.e., the hPSP expression level in healthy tissue from an individual not having the digestive, endocrine and immune system disorder. Thus, the invention provides a diagnostic method useful during diagnosis of such a disorder, which involves: (a) assaying hPSP gene expression level in cells or body fluid of an individual; (b) comparing the hPSP gene expression level with a standard hPSP gene expression level, whereby an increase or decrease in the assayed hPSP gene expression level compared to the standard expression level is indicative of disorder in the digestive, the endocrine, and the immmune systems.

An additional aspect of the invention is related to a method for treating an individual in need of an increased level of hPSP activity in the body comprising administering to such an individual a composition comprising a therapeutically effective amount of an isolated hPSP polypeptide of the invention or an agonist thereof.

A still further aspect of the invention is related to a method for treating an individual in need of a decreased level of hPSP activity in the body comprising, administering to such an individual a composition comprising a therapeutically effective amount of an hPSP antagonist. Preferred antagonists for use in the present invention are hPSP specific antibodies.

### BRIEF DESCRIPTION OF THE FIGURES

FIG. 1 shows the nucleotide sequence (SEQ ID NO: 1) and deduced amino acid sequence (SEQ ID NO: 2) of hPSP. The predicted leader sequence of about 18 amino acids is underlined. Two consensus N-linked glycosylation sites (NX(S/T)) appear in italics at positions corresponding to 107-109 (NLS) and 115-117 (NVT) of SEQ ID NO: 2.

FIG. 2 shows an alignment of the amino acid sequences of the hPSP protein and translation products of the mRNAs for mouse PSP (moPSP; SEQ ID NO: 3), rat PSP (ratPSP; SEQ ID NO: 4) and rat SMG-A (ratSMGA; SEQ ID NO: 5).

FIG. 3 shows an analysis of the hPSP amino acid sequence. Alpha, beta, turn and coil regions; hydrophilicity and hydrophobicity; amphipathic regions; flexible regions; antigenic index and surface probability are shown. In the “Antigenic Index—Jameson-Wolf” graph, the positive peaks indicate locations of the highly antigenic regions of the hPSP protein, i.e., regions from which epitope-bearing peptides of the invention can be obtained.

### DETAILED DESCRIPTION

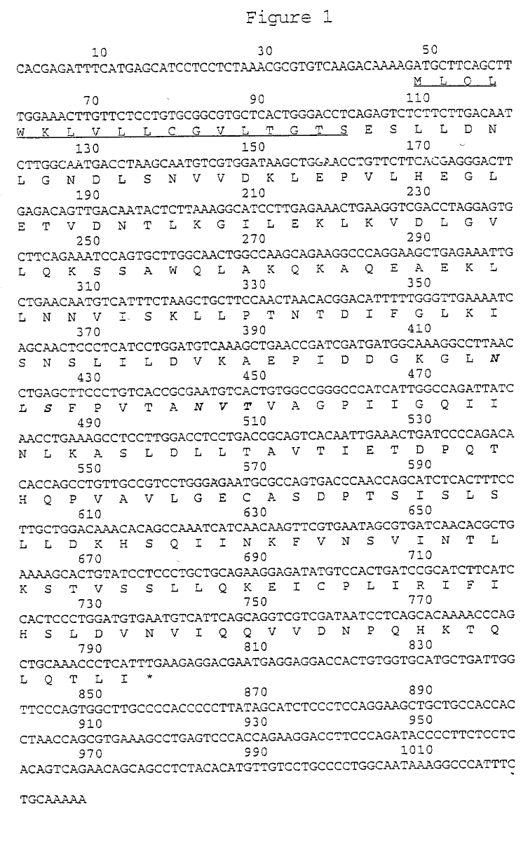

The present invention provides isolated nucleic acid molecules comprising a polynucleotide encoding a hPSP polypeptide having the amino acid sequence shown in FIG. 1 (SEQ ID NO: 2), which was determined by sequencing a cloned cDNA. The nucleotide sequence shown in FIG. 1 (SEQ ID NO: 1) was obtained by sequencing the HSGSA61 clone, which was deposited on Nov. 26, 1996 at the American Type Culture Collection, 12301 Park Lawn Drive, Rockville, Md. 20852, and given accession number ATCC 97811. The deposited clone is contained in the pBluescript SK(−) plasmid (Stratagene, La Jolla, Calif.).

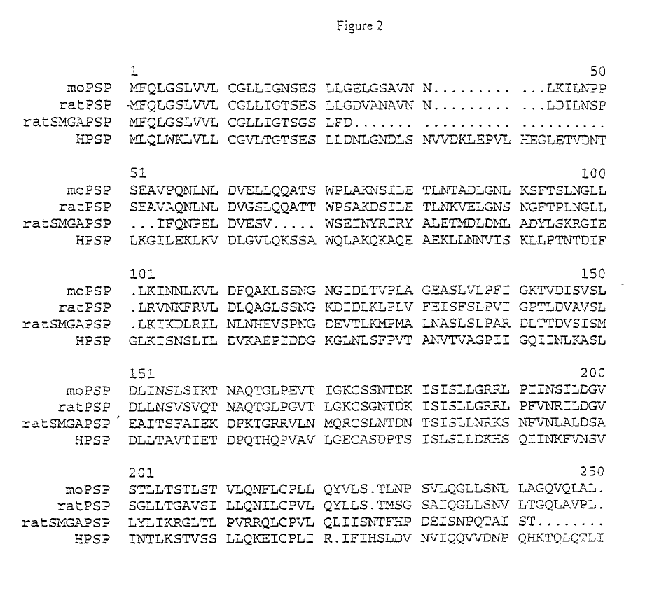

The hPSP protein of the present invention shares extensive sequence homology with three known murine members of the salivary gland secretory protein multigene family (Mirel & Ball, 1992, supra). The amino acid sequence of the hPSP protein shown in FIG. 1 (SEQ ID NO: 2) includes the conserved PSP region bounded by two Cys residues (residues 161 and 204 in mouse and rat PSP (Mirels & Ball, 1992, supra) and residues 174 and 217 in the complete human PSP sequence (i.e., amino acids 156 to 199 in SEQ ID NO:2). See FIG. 2. For instance, using the the computer program Bestfit (Wisconsin Sequence Analysis Package, Version 8 for Unix, Genetics Computer Group, University Research Park, 575 Science Drive, Madison, Wis. 53711) with the default parameters, the complete hPSP amino acid sequence (SEQ ID NO: 2) shares 33.6% identity and 57.9% similarity to the translation product of the mouse PSP mRNA (SEQ ID NO: 3; Madsen & Hjorth, 1985, supra, GenBank Accession No. X01697), 31.1% identity and 59.6% similarity with that of rat PSP mRNA (SEQ ID NO: 4), and 30.1% identity and 57.8% similarity with that of rat SMG-A mRNA (SEQ ID NO: 5; Mirels & Ball, 1992, supra; GenBank Accession No. M83210. Thus, the hPSP amino acid sequence is clearly a member of the PSP multigene family and is most closely related to the known murine PSP sequences. Therefore, this novel human protein has been designate human Parotid Secretory Protein (hPSP). However, hPSP is also highly similar to the rat SMG-A protein and may therefore represent a human homologue of this submandibular gland protein or of the related rat sublingual protein (Mirels & Ball, 1992, supra).

Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIG. 1 (SEQ ID NO: 1), a nucleic acid molecule of the present invention encoding hPSP polypeptide may be obtained using standard cloning and screening procedures, such as those for cloning cDNAs using mRNA as starting material. Illustrative of the invention, the nucleic acid molecule having the sequence described in FIG. 1 (SEQ ID NO: 1) was discovered in a cDNA library derived from human salivary gland tissue.

The determined nucleotide sequence of the hPSP cDNA of FIG. 1 (SEQ ID NO: 1) contains an open reading frame encoding a protein of 249 amino acid residues, with an initiation codon at nucleotide positions 49-51 of the nucleotide sequence in FIG. 1 (SEQ ID NO: 1), and a deduced molecular weight of about 27 kDa. As one of ordinary skill would appreciate, due to the possibilities of sequencing errors discussed above, the actual complete hPS polypeptide encoded by the deposited cDNA, which comprises about 249 amino acids, may be somewhat longer or shorter. More generally, the actual open reading frame may be anywhere in the range of ±20 amino acids, more likely in the range of ±10 amino acids, of that predicted from the coding sequence shown in FIG. 1 (SEQ ID NO: 1).

Leader and Mature Sequences

The amino acid sequence of the complete hPSP protein includes a leader sequence and a mature protein, as shown in FIG. 1 (SEQ ID NO: 2). More in particular, the present invention provides nucleic acid molecules encoding a mature form of the hPSP protein. Thus, according to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature hPSP polypeptide having the amino acid sequence encoded by the cDNA clone contained in the host identified as ATCC Deposit No. 9781 1. By the “mature hPSP polypeptide having the amino acid sequence encoded by the cDNA clone in ATCC Deposit No. 97811” is meant the mature form(s) of the hPSP protein produced by expression in a mammalian cell (e.g., COS cells, as described below) of the complete open reading frame encoded by the human DNA sequence of the clone contained in the vector in the deposited host.

The predicted sequence of 231 amino acids of the mature hPSP polypeptide is expected to yield an approximately 25 kDa band. Upon expression in a baculovirus expression system as decsribed hereinbelow, mutliple bands in the range of 25 to 31 kDA were observed. The molecules appearing to be larger than 25 kD may be explained by differential glycosylation and/or differential proteolytic degradation of the secreted protein. Evidence to support this conclusion includes the two consensus N-linked glycosylation sites present in the amino acid sequence (FIG. 1).

In addition, methods for predicting whether a protein has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequence of the complete hPSP polypeptide was analyzed by a computer program called PSORT (K. Nakai and M. Kanehisa, Genomics 14:897-911 (1992). The analysis of the hPSP amino acid sequence by this program supported the prediction of a leader cleavage site after the first 18 N-terminal residues (amino acids −18 to −1 of SEQ ID NO: 2) which was based on homology to the known leader sequence of mouse PSP.

It is expected, therefore, that while expression of hPSP in different eukaryotic cells may result in more than one species of mature protein that they will all be within three amino acids of the determined and predicted cleavage sites (i.e., the leader sequence could be between about 15 and 21 amino acids long and the mature protein could be between about 228 and 234 amino acids in length). More in particular it is predicted that most if not all mature species of the hPSP polypeptide of the invention have an amino acid sequence represented as follows: −3 to 231, −2 to 231, −1 to 231, to 231, 2 to 231, 3 to 231 and 4 to 231, all of SEQ ID NO: 2. Polynucleotides encoding such polypeptides are also provided.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 49-51 of the nucleotide sequence shown in FIG. 1 (SEQ ID NO: 1); i.e., nucleotides 49 to 795. Also included are DNA molecules comprising the coding sequence for the predicted mature hPSP protein shown in FIG. 1 (carboxy terminal 231 amino acids) (SEQ ID NO: 2).

In addition, isolated nucleic acid molecules of the invention include DNA molecules which comprise a sequence substantially different from those described above but which, due to the degeneracy of the genetic code, still encode the hPSP protein. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another aspect, the invention provides isolated nucleic acid molecules encoding the hPSP polypeptide having an amino acid sequence encoded by the cDNA clone contained in the plasmid deposited as ATCC Deposit No. 9781 ion Nov. 26, 1996. Preferably, this nucleic acid molecule will encode the mature polypeptide encoded by the above-described deposited cDNA clone.

The invention further provides an isolated nucleic acid molecule having the nucleotide sequence shown in FIG. 1 (SEQ ID NO: 1) or the nucleotide sequence of the hPSP cDNA contained in the above-described deposited clone, or a nucleic acid molecule having a sequence complementary to one of the above sequences. Such isolated molecules, particularly DNA molecules, are useful as probes for gene mapping, by in situ hybridization with chromosomes, and for detecting expression of the hPSP gene in human tissue, for instance, by Northern blot analysis.

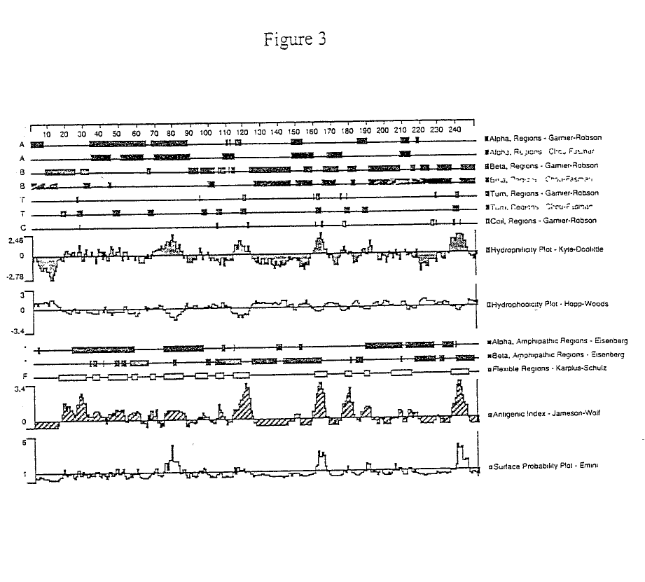

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO: 1 which consists of positions 49-855 of SEQ ID NO: 1. Further, the invention includes a polynucleotide comprising any portion of at least about 30 nucleotides, preferably at least about 50 nucleotides, of SEQ ID NO: 1 from residue 1 to 1028, more preferably, from positions 49 to 795 of SEQ ID NO: 1. More generally, by a fragment of an isolated nucleic acid molecule having the nucleotide sequence of the deposited cDNA or the nucleotide sequence shown in FIG. 1 (SEQ ID NO: 1) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50-300 nt in length are also useful according to the present invention as are fragments corresponding to most, if not all, of the nucleotide sequence of the deposited cDNA or as shown in FIG. 1 (SEQ ID NO: 1). By a fragment at least 20 nt in length, for example, is intended fragments which include 20 or more contiguous bases from the nucleotide sequence of the deposited cDNA or the nucleotide sequence as shown in FIG. 1 (SEQ ID NO: 1). Preferred nucleic acid fragments of the present invention include nucleic acid molecules encoding epitope-bearing portions of the hPSP polypeptide as identified in FIG. 3 and described in more detail below.

Several nucleic acid sequence which are related to the nucleic acid sequence shown in FIG. 1 (SEQ ID NO: 1) are shown in the sequence listing as follows: HSGSA61R(SEQ ID NO: 10); HSGSC13R(SEQ ID NO: 11); HSGSA89R (SEQ ID NO: 12); HSPAI14R (SEQ ID NO: 13); HSGSC78R (SEQ ID NO: 14); HSPMD56R (SEQ ID NO: 15); HSPMF91R (SEQ ID NO: 16); HSGSA3-R (SEQ ID NO: 17); and HSPMF57R (SEQ ID NO: 18). Preferred nucletc acid fragments of the invention comprise a polynucleotide sequence of at least 30 contiguous nucleotides, more preferrably at least 50 contiguous nucleotides, of SEQ ID NO: 1 wherein said fragment does not comprise any of SEQ ID NO: 10-18 or any subfragment of at least 30 contiguous nucleotides. preferrably at least 50 contiguous nucleotides, of any of SEQ ID NOS: 10-18.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 97811. By “stringent hybridization conditions” is intended overnight incubation at 42° C. in a solution comprising: 50%, formamide, 5× SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., about 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIG. 1 (SEQ ID NO: 1)). Of course, a polynucleotide which hybridizes only to a poly A sequence (such as the 3′ terminal poly(A) tract of the hPSP cDNA shown in FIG. 1 (SEQ ID NO: 1)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode an hPSP polypeptide may include, but are not limited to those encoding the amino acid sequence of the mature polypeptide, by itself; and the coding sequence for the mature polypeptide and additional sequences, such as those encoding the about 18 amino acid leader or secretory sequence, such as a pre-, or pro- or prepro- protein sequence; the coding sequence of the mature polypeptide, with or without the aforementioned additional coding sequences.

Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calf., 91311), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the hPSP fused to Fc at the N- or C-terminus.

Variant and Mutant Polynucleotides

The present invention further relates to variants of the nucleic acid molecules of the present invention, which encode portions, analogs or derivatives of the hPSP protein. Variants may occur naturally, such as a natural allelic variant. By an “alleic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the hPSP protein or portions thereof. Also especially preferred in this regard are conservative substitutions. Most highly preferred are nucleic acid molecules encoding the mature protein having the amino acid sequence shown in FIG. 1 (SEQ ID NO: 2) or the mature hPSP amino acid sequence encoded by the deposited cDNA clone.

Further embodiments include an isolated nucleic acid molecule comprising a polynucleotide having a nucleotide sequence at least 90% identical, and more preferably at least 95%, 96%, 97%, 98% or 99% identical to a polynucleotide selected from the group consisting of: (a) a nucleotide sequence encoding the hPSP polypeptide having the complete amino acid sequence in FIG. 1 (SEQ ID NO: 2); (b) a nucleotide sequence encoding the hPSP polypeptide having the complete amino acid sequence in FIG. 1 (SEQ ID NO: 2) excepting the N-terminal methionine; (c) a nucleotide sequence encoding the predicted mature hPSP polypeptide having the amino acid sequence at positions 1-231 in SEQ ID NO: 2; (d) a nucleotide sequence encoding the hPSP polypeptide having the complete amino acid sequence encoded by the cDNA clone contained in ATCC Deposit No. 97811 (e) a nucleotide sequence encoding the hPSP polypeptide having the complete amino acid sequence excepting the N-terminal methionine encoded by the cDNA clone contained in ATCC Deposit No. 97811; (f) a nucleotide sequence encoding the mature hPSP polypeptide having the amino acid sequence encoded by the cDNA clone contained in ATCC Deposit No. 97811; and (g) a nucleotide sequence complementary to any of the nucleotide sequences in (a), (b), (c), (d), (e) or (f) above.

By a polynucleotide having a nucleotide sequence at least, for example, 95% “identical” to a reference nucleotide sequence encoding a hPSP polypeptide is intended that the nucleotide sequence of the polynucleotide is identical to the reference sequence except that the polynucleotide sequence may include up to five point mutations per each 100 nucleotides of the reference nucleotide sequence encoding hPSP polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 1 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 5371 1). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%,95%,96%, 97%, 98% or 99% identical to the nucleic acid sequence shown in FIG. 1 (SEQ ID NO: 1) or to the nucleic acid sequence of the deposited cDNA, irrespective of whether they encode a polypeptide having hPSP activity. This is because even where a particular nucleic acid molecule does not encode a polypeptide having hPSP activity, one of skill in the art would still know how to use the nucleic acid molecule, for instance, as a hybridization probe or a polymerase chain reaction (PCR) primer. Uses of the nucleic acid molecules of the present invention that do not encode a polypeptide having hPSP activity include, inter alia, (1) isolating the hPSP gene or allelic variants thereof in a cDNA library; (2) in situ hybridization (e.g., “FISH”) to metaphase chromosomal spreads to provide precise chromosomal location of the hPSP gene, as described in Verma et al., Human Chromosomes: A Manual of Basic Techniques, Pergamon Press, New York (1988); and Northern Blot analysis for detecting hPSP mRNA expression in specific tissues.

Preferred, however, are nucleic acid molecules having sequences at least 90%, 95%, 96%, 97%, 98% or 99% identical to the nucleic acid sequence shown in FIG. 1 (SEQ ID NO: 1) or to the nucleic acid sequence of the deposited cDNA which do, in fact, encode a polypeptide having hPSP protein activity. By “a polypeptide having hPSP activity” is intended polypeptides exhibiting activity similar, but not necessarily identical, to an activity of the mature hPSP protein of the invention, as measured in a particular biological assay. Thus, “a polypeptide having hPSP protein activity” includes polypeptides that also exhibit any of the same activities as an hPSP polypeptide, such as binding to an antibody or receptor, in a dose-dependent manner. Although the degree of dose-dependent activity need not be identical to that of the hPSP protein, preferably, “a polypeptide having hPSP protein activity” will exhibit substantially similar dose-dependence in a given activity as compared to the hPSP protein (i.e., the candidate polypeptide will exhibit greater activity or not more than about 25-fold less and, preferably, not more than about tenfold less activity relative to the reference hPSP protein).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIG. 1 (SEQ ID NO: 1) will encode a polypeptide “having hPSP protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having hPSP protein activity. This is because the skilled artisan is fully aware of amino acid substitutions that are either less likely or not likely to significantly effect protein function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of hPSP polypeptides or fragments thereof by recombinant techniques. The vector may be, for example, a-phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc., supra; pBS vectors, Phagescript vectors, Bluescript vectors, pNH8A, pNH16a, pNH18A, pNH46A, available from Stratagene; and ptrc99a, pKK223-3, pKK233-3, pDR540, pRtIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995).

The hPSP protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

hPSP Polypeptides and Fragments

The invention further provides an isolated hPSP polypeptide having the amino acid sequence encoded by the deposited cDNA, or the amino acid sequence in FIG. 1 (SEQ ID NO: 2), or a peptide or polypeptide comprising a portion of the above polypeptides.

Variant and Mutant Polypeptides

To improve or alter the characteristics of hPSP polypeptides, protein engineering may be employed. Recombinant DNA technology known to those skilled in the art can be used to create novel mutant proteins or “muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terrninus without substantial loss of biological function. For instance, Ron et al., J. Biol. Chem., 268:2984-2988 (1993) reported modified KGF proteins that had heparin binding activity even if 3, 8, or 27 amino-terminal amino acid residues were missing. In the present case, since the protein of the invention is a member of the PSP polypeptide family, deletions of N-terminal amino acids up to the Leu at position 26 in SEQ ID NO: 2 may retain some biological activity.

However, even if deletion of one or more amino acids from the N-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the hPSP shown SEQ ID NO: 2, up to the Leu residue at position number 26, and polynucleotides encoding such polypeptides. In particular, the present invention provides polypeptides comprising the amino acid sequence of residues n-231 in SEQ ID NO: 2, where n is an integer except zero in the range of -17 to +26.

More in particular, the invention provides polynucleotides encoding polypeptides having the amino acid sequence of residues −17 to +231, −16 to +231, −15 to +231, −14 to +231, −13 to +231, −12 to +231, to +231, −10 to +231, −9 to +231, −8 to +231, −7 to +231, −6 to +231, −5 to +231, −4 to +231, −3 to +231, −2 to +231, −1 to +231, +1 to +231, +2 to +231, +3 to +231, +4 to +231, +5 to +231, +6 to +231, +7 to +231, +8 to +231, +9 to +231, +10 to +231, +11 to +231, +12 to +231, +13 to +231, +14 to +231, +15 to +231, +16 to +231, +17 to +231, +18 to +231, +19 to +231, +20 to +231, +21 to +231, +22 to +231, +23 to +231, +24 to +231, +25 to +231 and +26 to +231 of SEQ ID NO: 2. Polynucleotides encoding these polypeptides also are provided.

Similarly, many examples of biologically functional C-terminal deletion muteins are known. For instance, interferon gamma shows up to ten times higher activities by deleting 8-10 amino acid residues from the carboxy terminus of the protein (Dobeli et al., J. Biotechnology 7:199-216 (1988). In the present case, since the protein of the invention is a member of the PSP polypeptide family, deletions of C-terminal amino acids up to the Asn at position 220 in SEQ ID NO: 2, which is located at about the C-terminal end of a highly conserved region in the human and three murine members of the PSP multigene family showin in FIG. 2, may retain some biological activity

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of the hPSP shown in SEQ ID NO: 2, up to the Asn residue at position 220, and polynucleotides encoding such polypeptides. In particular, the present invention provides polypeptides having the amino acid sequence of residues 1-m of the amino acid sequence in SEQ ID NO: 2, where m is any integer in the range of 220-231.

More in particular, the invention provides polynucleotides encoding polypeptides having the amino acid sequence of residues 1-220, 1-221, 1-222, 1-223, 1-224, 1-225, 1-226, 1-227, 1-228, 1-229, 1-230 and 1-231 of SEQ ID NO: 2. Polynucleotides encoding these polypeptides also are provided.

The invention also provides polypeptides having one or more amino acids deleted from both the amino and the carboxyl termini, which may be described generally as having residues n-m of SEQ ID NO: 2, where n and m are integers as described above.

Also included are a nucleotide sequence encoding a polypeptide comprising a portion of the complete hPSP amino acid sequence encoded by the cDNA clone contained in ATCC Deposit No. 97811, where this portion excludes from 1 to about 43 amino acids from the amino terminus of the complete amino acid sequence encoded by the cDNA clone contained in ATCC Deposit No. 97811, or from 1 to about 11 amino acids from the carboxy terminus, or any combination of the above amino terminal and carboxy terminal deletions, of the complete amino acid sequence encoded by the cDNA clone contained in ATCC Deposit No. 97811. Polynucleotides encoding all of the above deletion mutant polypeptide forms also are provided.

Other Mutants

In addition to terminal deletion forms of the protein discussed above, it also will be recognized by one of ordinary skill in the art that some amino acid sequences of the hPSP polypeptide can be varied without significant effect of the structure or function of the protein. If such differences in sequence are contemplated, it should be remembered that there will be critical areas on the protein which determine activity.

Thus, the invention further includes variations of the hPSP polypeptide which show substantial hPSP polypeptide activity or which include regions of hPSP protein such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U. et al., supra, and the references cited therein. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of FIG. 1 (SEQ ID NO: 2), or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the hPSP of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutannne |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the hPSP protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard et al., Clin. Exp. Immunol. 2:331-340 (1967); Robbins et al., Diabetes 36: 838-845 (1987); Cleland et al, Crit. Rev. Therapeutic Drug Carrier Systems 10:307-377 (1993).

Replacement of amino acids can also change the selectivity of the binding of a ligand to cell surface receptors. For example, Ostade et al., Nature 361:266-268 (1993) describes certain mutations resulting in selective binding of 20 TNF-α to only one of the two known types of TNF receptors. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

As described above, hPSP contains the two Cys residues and intevening region (i.e., amino acids 166 to 199 in SEQ ID NO: 2) conserved in the three murine members of the PSP multigene family shown in FIG. 2. Therefore, to modulate rather than completely eliminate biological activities of hPSP preferably mutations are made in sequences encoding amino acids in this hPSP conserved domain, more preferably in residues within this region which are not conserved in all members of the PSP. Also forming part of the present invention are isolated polynucleotides comprising nucleic acid sequences which encode the above hPSP mutants.

The polypeptides of the present invention are preferably provided in an isolated form, and preferably are substantially purified. A recombinantly produced version of the hPSP polypeptide can be substantially purified by the one-step method described in Smith and Johnson, Gene 67:31-40 (1988). Polypeptides of the invention also can be purified from natural or recombinant sources using anti-hPSP antibodies of the invention in methods which are well known in the art of protein purification.

The invention further provides an isolated hPSP polypeptide comprising an amino acid sequence selected from the group consisting of: (a) the amino acid sequence of the full-length hPSP polypeptide having the complete amino acid sequence shown in SEQ ID NO: 2 or the complete amino acid sequence encoded by the cDNA clone contained in the ATCC Deposit No. 97811 (b) the amino acid sequence of the full-length hPSP polypeptide having the complete amino acid sequence shown in SEQ ID NO: 2 excepting the N-terminal methionine (i.e., positions −17 to 231 of SEQ ID NO: 2) or the complete amino acid sequence excepting the N-terminal methionine encoded by the cDNA clone contained in the ATCC Deposit No. 9781 1; (c) the amino acid sequence of the mature hPSP shown in SEQ ID NO: 2 at positions 1 to 231 or the amino acid sequence of the mature hPSP polypeptide encoded by the cDNA clone contained in the ATCC Deposit No. 97811.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNA or to the polypeptide of FIG. 1 (SEQ ID NO: 2), and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a hPSP polypeptide is intended that the amino acid sequence of the polypeptide is identical to the reference sequence except that the polypeptide sequence may include up to five amino acid alterations per each 100 amino acids of the reference amino acid of the hPSP polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIG. 1 (SEQ ID NO: 2) or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting hPSP protein expression as described below or as agonists and antagonists capable of enhancing or inhibiting hPSP protein function. Further, such polypeptides can be used in the yeast two-hybrid system to “capture” hPSP protein binding proteins which are also candidate agonists and antagonists according to the present invention. The yeast two hybrid system is described in Fields and Song, Nature 340:245-246 (1989).

Epitope-Bearing Portions

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998- 4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green. N. and Learner, R. A. (1983) “Antibodies that react with predetermined sites on proteins,” Science, 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to hPSP specific antibodies include: a polypeptide comprising amino acid residues from about Ser50 to about Leu66 of SEQ ID NO: 2; a polypeptide comprising amino acid residues from about Glu97 to about Leu105 of SEQ ID NO: 2; a polypeptide comprising amino acid residues from about Glu141 to about Gln148 of SEQ ID NO: 2; and a polypeptide comprising amino acid residues from about Asp2l9 to about Leu227 of SEQ ID NO: 2. These polypeptide fragments have been determined to bear antigenic epitopes of the hPSP protein by the analysis of the Jameson-Wolf antigenic index, as shown in FIG. 3, above.

The epitope-bearing peptides and polypeptides of the invention may be produced by any conventional means. See, e.g., Houghten, R. A. (1985) “General method for the rapid solid-phase synthesis of large numbers of peptides: specificity of antigen-antibody interaction at the level of individual amino acids.” Proc. Natl. Acad. Sci. USA 82:5131-5135; this “Simultaneous Multiple Peptide Synthesis (SMPS)” process is further described in U.S. Pat. No. 4,631,211 to Houghten et al. (1986).

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985). Immunogenic epitope-bearing peptides of the invention, i.e., those parts of a protein that elicit an antibody response when the whole protein is the immunogen, are identified according to methods known in the art. See, for instance, Geysen et al., supra. Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Fusion Proteins

As one of skill in the art will appreciate, hPSP polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric hPSP protein or protein fragment alone (Fountoulakis et al., J. Biochem. 270:3958-3964 (1995)).

Digestive, Nonimmune Defense, Endocrine and Immune System-Related Disorder Diagnosis

The present inventors have discovered that mRNA related to the hPSP cDNA cloned from salivary gland tissue is highly expressed in human salivary gland tissue and related mRNA to a much lesser extent in pancreas and thymus. Given the involvement of these tissues in the digestive, nonimmune defense, endocrine and immune systems, for a number of disorders related to these systems substantially altered (increased or decreased) levels of hPSP gene expression can be detected in tissue or other cells or bodily fluids (e.g., sera, plasma, urine, synovial fluid or spinal fluid) taken from an individual having such a disorder, relative to a “standard” hPSP gene expression level, that is, the hPSP expression level in above tissues or bodily fluids from an individual not having a disorder of the above systems. Thus, the invention provides a diagnostic method useful during diagnosis of a digestive, an endocrine, or an immune system disorder, which involves measuring the expression level of the gene encoding the hPSP protein in digestive a, endocrine, or immune system tissue or other cells or body fluid from an individual and comparing the measured gene expression level with a standard hPSP gene expression level, whereby an increase or decrease in the gene expression level compared to the standard is indicative of an digestive, an endocrine, or an immune system disorder.

In particular, it is believed that certain tissues in mammals with cancer of the salivary gland, the thymus or the pancreas express significantly increased levels of the hPSP protein and mRNA encoding the hPSP protein when compared to a corresponding “standard” level. See, for instance, Prasad, K.

N., et al., supra, in which the authors particularly noted that the level of PSP, measured with both immunological and nucleic acid hybridization methods with mouse PSP reagents, increased upon transformation of human nontumorigenic parotid gland acinar cells to cancer cells. Further, it is believed that enhanced levels of the hPSP protein can be detected in certain body fluids (e.g., particularly saliva, but also sera, plasma, urine, and spinal fluid) from mammals with such a cancer when compared to sera from mammals of the same species not having the cancer.

Thus, the invention provides a diagnostic method useful during diagnosis of a digestive, nonimmune defense, endocrine or immune system disorder, including cancers of these systems, which involves measuring the expression level of the gene encoding the hPSP protein in a tissue of such a system or other cells or body fluid from an individual and comparing the measured gene expression level with a hPSP gene expression level, whereby an increase or decrease in the gene expression level compared to the standard is indicative of a digestive, nonimmune defense, endocrine or immune system disorder. Where a diagnosis of a disorder in the digestive, an endocrine or an immune system, including diagnosis of a tumor, has already been made according to conventional methods, the present invention is useful as a prognostic indicator, whereby patients exhibiting enhanced or reduced hPSP gene expression will experience a worse clinical outcome relative to patients expressing the gene at a level nearer the standard level.

By “assaying the expression level of the gene encoding the hPSP protein” is intended qualitatively or quantitatively measuring or estimating the level of the hPSP protein or the level of the mRNA encoding the hPSP protein in a first biological sample either directly (e.g., by determining or estimating absolute protein level or mRNA level) or relatively (e.g., by comparing to the hPSP protein level or mRNA level in a second biological sample). Preferably, the hPSP protein level or mRNA level in the first biological sample is measured or estimated and compared to a standard hPSP protein level or mRNA level, the standard being taken from a second biological sample obtained from an individual not having the disorder or being determined by averaging levels from a population of individuals not having a disorder of the digestive, nonimmune defense, endocrine, or immune system. As will be appreciated in the art, once a standard hPSP protein level or mRNA level is known, it can be used repeatedly as a standard for comparison.

By “biological sample” is intended any biological sample obtained from an individual, body fluid, cell line, tissue culture, or other source which contains hPSP protein or mRNA. As indicated, biological samples include body fluids (such as sera, plasma, urine, synovial fluid and spinal fluid) which contain free hPSP protein, digestive, endocrine, or immune system tissue, and other tissue sources found to express complete or mature form of the hPSP or a hPSP receptor. Methods for obtaining tissue biopsies and body fluids from mammals are well known in the art. Where the biological sample is to include mRNA, a tissue biopsy is the preferred source.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987), Levels of mRNA encoding the hPSP protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying hPSP protein levels in a biological sample can occur using antibody-based techniques. For example, hPSP protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting hPSP protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying hPSPprotein levels in a biological sample obtained from an individual, hPSPprotein can also be detected in vivo by imaging. Antibody labels or markers for in vivo imaging of hPSP protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

An hPSP protein-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain hPSP protein. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)).

Antibodies

hPSP protein specific antibodies for use in the present invention can be raised against the intact hPSP protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)2 fragments) which are capable of specifically binding to hPSP protein. Fab and F(ab′)2 fragments lack the Fc fragment of intact antibody, clear more rapidly from the circulation, and may have less non-specific tissue binding of an intact antibody (Wahl er al., J. Nucl. Med. 24:316-325 (1983)). Thus, these fragments are preferred.

The antibodies of the present invention may be prepared by any of a variety of methods. For example, cells expressing the hPSP protein or an antigenic fragment thereof can be administered to an animal in order to induce the production of sera containing polyclonal antibodies. In a preferred method, a preparation of hPSP protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or hPSP protein binding fragments thereof). Such monoclonal antibodies can be prepared using hybridoma technology (Kohler et al., Nature 256:495 (1975); Kohler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., (1981) pp. 563-681 ). In general, such procedures involve immunizing an animal (preferably a mouse) with a hPSP protein antigen or, more preferably, with a hPSP protein-expressing cell. Suitable cells can be recognized by their capacity to bind anti-hPSP protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Rockville, Md. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the hPSP protein antigen.

Alternatively, additional antibodies capable of binding to the hPSP protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, hPSP ]-protein specific antibodies are used to immunize an animal, preferably a mouse. The splenocytes of such an animal are then used to produce hybridoma cells, and the hybridoma cells are screened to identify clones which produce an antibody whose ability to bind to the hPSP protein-specific antibody can be blocked by the hPSP protein antigen. Such antibodies comprise anti-idiotypic antibodies to the hPSP protein-specific antibody and can be used to immunize an animal to induce formation of further hPSP protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, hPSP protein-binding fragments can be produced through the application of recombinant DNA technology or through synthetic chemistry.

For in vivo use of anti-hPSP in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

Treatment of Digestive, Nonimmune Defense, Endocrine or Immune System-Related Disorders

As noted above, hPSP polynucleotides and polypeptides are useful for diagnosis of conditions involving abnormally high or low expression hPSP activities. Given the cells and tissues where hPSP (or proteins of the hPSP family) is expressed (salivary gland, pancreas and thymus), it is readily apparent that a substantially altered (increased or decreased) level of expression of hPSP in an individual compared to the standard or “normal” level produces pathological conditions related to the bodily system(s) in which hPSP is expressed and/or is active.

It will also be appreciated by one of ordinary skill that, since the hPSP protein of the invention is a member of the PSP mature form of the protein may be released in soluble form from the cells which express the hPSP by proteolytic cleavage, i.e., either into the saliva, if from salivary gland cells, or into the digestive tract or systemically, if produced by the pancreas or thymus Therefore, when mature hPSP polypeptide is added from an exogenous source to cells, tissues or the body of an individual, the protein will exert its physiological activities on its target cells of that individual.

Therefore, it will be appreciated that conditions caused by a decrease in the standard or normal level of hPSP activity in an individual, particularly disorders of the digestive, nonimmune defense, endocrine, or immune system, can be treated by administration of hPSP polypeptide (in the form of the mature protein . Thus, the invention also provides a method of treatment of an individual in need of an increased level of hPSP activity comprising administering to such an individual a pharmaceutical composition comprising an amount of an isolated hPSP polypeptide of the invention, particularly a mature form of the hPSP protein of the invention, effective to increase the hPSP activity level in such an individual.

Formulations

The hPSP polypeptide composition will be formulated and dosed in a fashion consistent with good medical practice, taking into account the clinical condition of the individual patient (especially the side effects of treatment with hPSP polypeptide alone), the site of delivery of the hPSP polypeptide composition, the method of administration, the scheduling of administration, and other factors known to practitioners. For conditions in which the level of hPSP polypeptide in the saliva is determined to be below the standard level, hPSP polypeptide, preferably the mature form, is administered orally in amounts comparable to those normally produced in saliva. The “effective amount” of hPSP polypeptide for purposes herein is thus determined by the above considerations.

As a general proposition, the total pharmaceutically effective amount of hPSP polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the hPSP polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the hPSP of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The hPSP polypeptide is also suitably administered by sustained-release systems. Suitable examples of-sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D- (−)-3-hydroxybutyric acid (EP 133,988). Sustained-release hPSP polypeptide compositions also include liposomally entrapped hPSP polypeptide. Liposomes containing hPSP polypeptide are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal hPSP polypeptide therapy.

For parenteral administration, in one embodiment, the hPSP polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the hPSP polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The hPSP polypeptide is typically formulated in such vehicles at a concentration of about 0. 1 mg/ml to 100 mg/ml, preferably 1-10 mg/ml, at a pH of about 3 to 8. It will be understood that the use of certain of the foregoing excipients, carriers, or stabilizers will result in the formation of hPSP polypeptide salts.

hPSP polypeptide to be used for therapeutic administration must be sterile. Sterility is readily accomplished by filtration through sterile filtration membranes (e.g., 0.2 micron membranes). Therapeutic hPSP polypeptide compositions generally are placed into a container having a sterile access port, for example, an intravenous solution bag or vial having a stopper pierceable by a hypodermic injection needle.

hPSP polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous hPSP polypeptide solution, and the resulting mixture is lyophilized. The infusion solution is prepared by reconstituting the lyophilized hPSP polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of hPSP on cells, such as its interaction with hPSP-binding molecules such as receptor molecules. An agonist is a compound which increases the natural biological functions of hPSP or which functions in a manner similar to hPSP, while antagonists decrease or eliminate such functions.

In another aspect of this embodiment the invention provides a method for identifying a receptor protein or other ligand-binding protein which binds specifically to a hPSP polypeptide. For example, a cellular compartment, such as a membrane or a preparation thereof, may be prepared from a cell that expresses a molecule that binds hPSP. The preparation is incubated with labeled hPSP and complexes of hPSP bound to the receptor or other binding protein are isolated and characterized according to routine methods known in the art. Alternatively, the hPSP polypeptide may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds hPSP, such as a molecule of a signaling or regulatory pathway modulated by hPSP. The preparation is incubated with labeled hPSP in the absence or the presence of a candidate molecule which may be a hPSP agonist or antagonist. The ability of the candidate molecule to bind the binding molecule is reflected in decreased binding of the labeled ligand. Molecules which bind gratuitously, i.e., without inducing the effects of hPSP on binding the hPSP binding molecule, are most likely to be good antagonists. Molecules that bind well and elicit effects that are the same as or closely related to hPSP are agonists.

hPSP-like effects of potential agonists and antagonists may by measured, for instance, by determining activity of a second messenger system following interaction of the candidate molecule with a cell or appropriate cell preparation, and comparing the effect with that of hPSP or molecules that elicit the same effects as hPSP. Second messenger systems that may be useful in this regard include but are not limited to AMP guanylate cyclase, ion channel or phosphoinositide hydrolysis second messenger systems.

Another example of an assay for hPSP antagonists is a competitive assay that combines hPSP and a potential antagonist with membrane-bound hPSP receptor molecules or recombinant hPSP receptor molecules under appropriate conditions for a competitive inhibition assay. hPSP can be labeled, such as by radioactivity, such that the number of hPSP molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing hPSP-induced activities, thereby preventing the action of hPSP by excluding hPSP from binding.

Other potential antagonists include antisense molecules. Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of hPSP. The antisense RNA oligonucleotide hybridizes to the mRNA in vivo and blocks translation of the mRNA molecule into hPSP polypeptide. The oligonucleotides described above can also be delivered to cells such that the antisense RNA or DNA may be expressed in vivo to inhibit production of hPSP protein.

The agonists and antagonists may be employed in a composition with a pharmaceutically acceptable carrier, e.g., as described above.

The antagonists may be employed for instance to inhibit the biological activity of hPSP in the digestive, the endocrine, or the immune systems. Any of the above antagonists may be employed in a composition with a pharmaceutically acceptable carrier, e.g., as hereinafter described.

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a hPSP protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

Example 1(a): Expression and Purification of “His-tagged” hPSP in E. coli

The bacterial expression vector pQE60 is used for bacterial expression in this example (QIAGEN, Inc., 9259 Eton Avenue, Chatsworth, Calif., 9131 1). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that an inserted DNA fragment encoding a polypeptide expresses that polypeptide with the six His residues (i.e., a “6× His tag”) covalently linked to the carboxyl terminus of that polypeptide.

The DNA sequence encoding the desired portion hPSP protein lacking the hydrophobic leader sequence is amplified from the deposited cDNA clone using PCR oligonucleotide primers which anneal to the amino terminal sequences of the desired portion of the hPSP protein and to sequences in the deposited construct 3′ to the cDNA coding sequence. Additional nucleotides containing restriction sites to facilitate cloning in the pQE60 vector are added to the 5′and 3′ sequences, respectively.

For cloning the mature protein, the 5′primer has the sequence 5′ CTA CAG CCA TGG AGT CTC TTC TTG ACA ATC TTG GCA ATG 3′ (SEQ ID NO: 6) containing the underlined Nco I restriction site followed by 27 nucleotides of the amino terminal coding sequence of the mature hPSP sequence in FIG. 1. One of ordinary skill in the art would appreciate, of course, that the point in the protein coding sequence where the 5′primer begins may be varied to amplify a DNA segment encoding any desired portion of the complete protein shorter or longer than the mature form. The 3′ primer has the sequence 5′ CAT CGC GGA TCC AAT GAG GGT TTG CAG CTG GGT TTT G3′ (SEQ ID NO: 7) containing the underlined Bam HI restriction site followed by 25 nucleotides complementary to the 3′ end of the coding sequence immediately before the stop codon in the hPSP DNA sequence in FIG. 1, with the coding sequence aligned with the restriction site so as to maintain its reading frame with that of the six His codons in the pQE60 vector.

The amplified hPSP DNA fragment and the vector pQE60 are digested with Nco I and the digested DNAs are then ligated together. Insertion of the hPSP DNA into the restricted pQE60 vector places the hPSP protein coding region downstream from the IPTG-inducible promoter and in-frame with an initiating AUG and the six histidine codons.

The ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing hPSP protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. Isopropyl-β-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6M guanidine-HCl, pH 8. The cell debris is removed by centrifugation, and the supernatant containing the hPSP is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist, 1995, QIAGEN, Inc., supra). Briefly the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the hPSP is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 25 mM Tris/HCl pH 7.4, containing protease inhibitors. The renaturation should be performed over a period of 1.5 hours or more. After renaturation the proteins can be eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

If production of the hPSP mature polypeptide with no terminal “His tag” is desried in E. coli, one of ordinary skill would appreciate that the foregoing example may be modified by inclusion of the stop codon in the 3′ primer at the C-terminal end of the hPSP codiing sequence, so that the six His codons in the vector are not translated. In that event, the protein is produced as described above except for use of the His-tag for purification. For example, the cells containing expressed hPSP polypeptide are stirred for 3-4 hours at 4° C. in 6M guanidine-HCl, pH 8. The cell debris is removed by centrifugation, and the supernatant containing the hPSP is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure hPSP protein. The purified protein is stored at 4° C. or frozen at −80° C.

Alternatively, a preferred bacterial expression vector “pHE4-5” containing an neomycin phosphotransferase gene for selection may be used in this example. The “pHE4-5/MPIFΔ23” vector plasmid DNA contains a filler insert (MPIFΔ23) between unique restriction enzyme sites NdeI and Asp718 and was deposited with the American Type Culture Collection, 12301 Park Lawn Drive, Rockville, Md. 20852, on Sep. 30, 1997 and given Accession No. 209311. Using 5′and 3′ primers described herein with restriction enzyme sites for NdeI and Asp 718 substituted for the NcoI and HindIII sites in the respective primers, a suitable hPSP encoding DNA fragment for subcloning into pHE4-5 can be amplifed. The stuffer DNA insert in pHE⁴-5/MPIFΔ23 should be removed prior to ligating the hPSP fragment to pHE4-5. pHE4-5 contains a strong bacterial promoter allowing for high yields of most heterologous proteins.

### EXAMPLE 2

Cloning and Expression of hPSP protein in a Baculo virus Expression System

In this illustrative example, the plasmid shuttle vector pA2 is used to insert the cloned DNA encoding complete protein, including its naturally associated secretory signal (leader) sequence, into a baculovirus to express the mature hPSP protein, using standard methods as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The cDNA sequence encoding the full length hPSP protein in the deposited clone, including the AUG initiation codon and the naturally associated leader sequence shown in FIG. 1 (SEQ ID NO: 2), is amplified using PCR oligonucleotide primers corresponding to the 5′and 3′ sequences of the gene. The 5′primer has the sequence 5° CTA CGC GGA TCC GCC ATC ATG CTT CAG CTT TGG AAA CTT GTT C 3′ (SEQ ID NO: 8) containing the underlined Bam HI restriction enzyme site, an efficient signal for initiation of translation in eukaryotic cells, as described by Kozak, M., J. Mol. Biol. 196:947-950 (1987), followed by 25 nucleotides of the sequence of the complete hPSP protein shown in FIG. 1, beginning with the AUG initiation codon. The 3′ primer has the sequence 5′ CTC TGC TCT AGA CTA AAT GAG GGT TTG CAG C 3′ (SEQ ID NO: 9) containing the underlined Xba I restriction site followed by 16 nucleotides complementary to the 3′ coding sequence in FIG. 1 including two bases of the stop codon for which the first base is included in the Xba I restriction site.

The amplified fragment is isolated from a 1% agarose gel using a commercially available kit (“Geneclean,” BIO 101 Inc., La Jolla, Calif.). The fragment then is digested with Bam HI and Xba I and again is purified on a 1% agarose gel. This fragment is designated herein F1. The plasmid is digested with the restriction enzymes Bam HI and Xba I and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “VI”.

Fragment F1 and the dephosphorylated plasmid V1 are ligated together with T4 DNA ligase. E. coli HB 101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human hPSP gene by digesting DNA from individual colonies using Bam HI and Xba I and then analyzing the digestion product by gel electrophoresis. The sequence of the cloned fragment is confirmed by DNA sequencing. This plasmid is designated herein pA2hPSP.

Five μg of the plasmid pA2hPSP is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). One μg of BaculoGold™ virus DNA and 5 μg of the plasmid pA2hPSP are mixed in a sterile well of a microtiter plate containing 50 μl of serum-free Grace's medium (Life Technologies Inc., Gaithersburg, Md.). Afterwards, 10 μl ipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-hPSP.

To verify the expression of the hPSP gene Sf9 cells are grown in Grace's medium supplemented with 10% heat-inactivated FBS. The cells are infected with the recombinant baculovirus V-hPSP at a multiplicity of infection (“MOI”) of about 2. If radiolabeled proteins are desired, 6 hours later the medium is removed and is replaced with SF900 II medium minus rethionine and cysteine (available from Life Technologies Inc., Rockville, Md.)). After 42 hours, 5 ,μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of mature form of the hPSP protein and thus the cleavage point and length of the naturally associated secretory signal peptide.

### EXAMPLE 3

Cloning and Expression of hPSP in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′- intron, the polyadenylation and termination signal of the rat preproinsulin gene.

### EXAMPLE 3(a)

Cloning and Expression in COS Cells

The expression plasmid, phPSP HA, is made by cloning a portion of the cDNA encoding the mature form of the hPSP protein into the expression vector pcDNAI/Amp or pcDNAIII (which can be obtained from Invitrogen, Inc.). The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37: 767 (1984). The fusion of the HA tag to the target protein allows easy detection and recovery of the recombinant protein with an antibody that recognizes the HA epitope. pcDNAIII contains, in addition, the selectable neomycin marker.

A DNA fragment encoding mature hPSP polypeptide is cloned into the polylinker region of the vector so that recombinant protein expression is directed by the CMV promoter. The plasmid construction strategy is as follows. The hPSP cDNA of the deposited clone is amplified using primers that contain convenient restriction sites, much as described above for construction of baculovirus vectors for expression of hPSP in insect cells. Suitable 5′ primers include a convenient restriction site for the vector, a Kozak and a sequence of 15-25 nucleotides of the 5′coding region of the complete hPSP polypeptide beginning with the AUG intiation codon (at position 48 in SEQ ID NO: 1). Suitable 3′ primers contain a restriction site convenient for the vector and 15-20 nucleotides complementary to the 3′ coding sequence immediately before the stop codon.

The PCR amplified DNA fragment and the vector, pcDNAI/Amp, are digested with appropriate restriction enzymes and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the mature hPSP polypeptide

For expression of recombinant hPSP polypeptide, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of hPSP the vector.

Expression of the hPSP-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### EXAMPLE 3(b)

Cloning and Expression in CHO Cells

The vector pC4 is used for the expression of hPSP polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasnaid pC⁴ contains for expressing the gene of interest the strong promoter of the long terminal repeat (LTR) of the Rouse Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are the following single restriction enzyme cleavage sites that allow the integration of the genes: BamHI, Xba I, and Asp7 18. Behind these cloning sites the plasmid contains the 3′ intron and polyadenylation site of the rat preproinsulin gene. Other high efficiency promoters can also be used for the expression, e.g., the human B-actin promoter, the SV40 early or late promoters or the long terminal repeats from other retroviruses, e.g., HIV and HTLVI. Clontech's Tet-Off and Tet-On gene expression systems and similar systems can be used to express the hPSP polypeptide in a regulated way in mammalian cells (Gossen, M., & Bujard, H. 1992, Proc. Natl. Acad. Sci. USA 89:5547-5551). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Xba I and then dephosphorylated using calf intestinal phosphates by procedures known in the art. The vector is then isolated from a 1% agarose gel.

The DNA sequence encoding the complete hPSP polypeptide is amplified using PCR oligonucleotide primers corresponding to the 5′ and 3′ sequences of the desired portion of the gene. The 5′ primer containing the underlined Bam HI site, a Kozak sequence and 25 nucleotides of the 5′coding region of the complete hPSP polypeptide beginning with the AUG initiation codon, has the following sequence: 5′ CTA CGC GGA TCC GCC ATC ATG CTT CAG CTT TGG AAA CTT GTT C 3′ (SEQ ID NO: 8). The 3′ primer, containing the underlined Xba I and 16 of nucleotides complementary to the 3′ coding sequence followed by 16 nucleotides complementary to the 3′ coding sequence in FIG. 1 including two bases of the stop codon for which the first base is included in the Xba I restriction site, has the following sequence: 5′ CTC TGC TCT AGA CTA AAT GAG GGT TTG CAG C 3′ (SEQ ID NO: 9).

The arnplified fragment is digested with the endonucleases Bam HI and Xba I and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/mil G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100 - 200 μM. Expression of the desired gene product is analyzed, for instance, by SDS-PAGE and Western blot or by reversed phase HPLC analysis.

### EXAMPLE 4

Tissue distribution of hPSP mRNA expression

Northern blot analysis is carried out to examine hPSP gene expression in human tissues, using methods described by, among others, Sambrook et al., cited above. A cDNA probe containing the entire nucleotide sequence of the hPSP protein (SEQ ID NO: 1) is labeled with ³²p using the rediprime™ DNA labeling system (Amersham Life Science), according to manufacturer's instructions. After labeling, the probe is purified using a NucTrap Probe purification column (Stratagene, 400702) according to manufacturer's protocol number PT1200-1. The purified labeled probe is then used to examine various human tissues for hPSP mRNA.

Multiple Tissue Northern (MTN) blots containing various human tissues (I and II), human immune system tissues (IM), and human endocrine system tissues (En) were obtained from Clontech and were examined with the ³²P-labeled hPSP cDNA probe under stringent hybridization conditions. Briefly, a Northern blot filter was prehybridized in 10 ml of Hybrisol I solution (Oncor, S4040) for 3 hours at 42° C. Probe DNA was denatured and added to hybridization solution at 106 cpm/ml of solution. Hybridization was carried out at 42° C. overnight. The filter was washed for 10 minutes in 2×SSC containing 0.1% SDS at room temperature, 15 minutes in 0.2×SSC with 0.1% SDS at 45° C., and 10 minutes in 0.1×SSC with 0.1% SDS at 55° C. The filter was exposed to autoradiographic film (Amersham Hyperfilm-MP, RPN 1675) overnight at −80° C. Of all tissues tested in these blots, the only positive hybridization to hPSP-related mRNA was observed on the MTN blots only in the human salivary gland samples. Weak hybridization to pancreas and thymus samples was also observed be may be explained by cross-hybridization to a related family member of hPSP. Accordingly, it is believed that expression of hPSP is restricted to the salivary gland.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the hPSP polypeptide having the amino acid sequence at positions −18 to +231 in SEQ ID NO: 2;
(b) a nucleotide sequence encoding the hPSP polypeptide having the amino acid sequence at positions −17 to +231 in SEQ ID NO: 2;
(c) a nucleotide sequence encoding the predicted mature hPSPpolypeptide having the amino acid sequence at positions +1 to +231 in SEQ ID NO: 2;
(d) a nucleotide sequence encoding the hPSP polypeptide having the complete amino acid sequence encoded by the cDNA clone contained in ATCC Deposit No. 97811;
(e) a nucleotide sequence encoding the hPSP polypeptide having the complete amino acid sequence exceptin, the N-terminal methionine encoded by the cDNA clone contained in ATCC Deposit No. 97811;
(f) a nucleotide sequence encoding the mature hPSP polypeptide having the amino acid sequence encoded by the cDNA clone contained in ATCC Deposit No. 97811; and
(g) a nucleotide sequence complementary to any of the nucleotide sequences in (a), (b), (c), (d), (e) or (f) above.

2. The nucleic acid molecule of claim 1 wherein said polynucleotide has the complete nucleotide sequence in FIG. 1 (SEQ ID NO: 1).

3. The nucleic acid molecule of claim 1 wherein said polynucleotide has the nucleotide sequence in SEQ ID NO: 1 encoding the hPSP polypeptide having the amino acid sequence in positions −17 to +231 SEQ ID NO: 2.

4. The nucleic acid molecule of claim 1 wherein said polynucleotide has the nucleotide sequence in SEQ ID NO: 1 encoding the mature form of the hPSP polypeptide having the amino acid sequence from about amino acid residue 1 to about amino acid residue 231 of SEQ ID NO: 2.

5. An isolated nucleic acid molecule comprising a polynucleotide having a nucleotide sequence at least 95% identical to a sequence selected from the group consisting of:
(a) a nucleotide sequence encoding a polypeptide comprising the amino acid sequence at positions n-231 in SEQ ID NO: 2, where n is an integer except zero in the range of −17 to +26.
(b) a nucleotide sequence encoding a polypeptide having the amino acid sequence of residues 1-m of SEQ ID NO: 2, where n and m is an integer in the range of +220 to +231;
(c) a nucleotide sequence encoding a polypeptide having the amino acid sequence of residues n-m of SEQ ID NO: 2, where n and m are integers as defined respectively in (a) and (b) above; and
(d) a nucleotide sequence encoding a polypeptide consisting of a portion of the complete hPSP amino acid sequence encoded by the cDNA clone contained in ATCC Deposit No. 97811 wherein said portion excludes from 1 to about 43 amino acids from the amino terminus of said complete amino acid sequence encoded by the cDNA clone contained in ATCC Deposit No. 9781 1;
(e) a nucleotide sequence encoding a polypeptide consisting of a portion of the complete hPSP amino acid sequence encoded by the cDNA clone contained in ATCC Deposit No. 97811 wherein said portion excludes or from 1 to about 11 amino acids from the carboxy terminus of said complete amino acid sequence encoded by the cDNA clone contained in ATCC Deposit No. 97811; and
(f) a nucleotide sequence encoding a polypeptide comprisng a portion of the complete hPSP amino acid sequence encoded by the cDNA clone contained in ATCC Deposit No. 97811 wherein said portion include a combination of any of the amino terminal and carboxy terminal deletions in (d) and (e), above.

6. The nucleic acid molecule of claim 1 wherein said polynucleotide has the complete nucleotide sequence of the cDNA clone contained in ATCC Deposit No. 97811.

7. The nucleic acid molecule of claim 1 wherein said polynucleotide has the nucleotide sequence encoding the hPSP polypeptide having the complete amino acid sequence except the N-terminal amino acid encoded by the cDNA clone contained in ATCC Deposit No. 9781 1.

8. The nucleic acid molecule of claim 1 wherein said polynucleotide has the nucleotide sequence encoding the mature form of the hPSP polypeptide having the amino acid sequence encoded by the cDNA clone contained in ATCC Deposit No. 97811.

9. An isolated nucleic acid molecule comprising a nucleic acid sequence which encodes an epitope-bearing portion of an hPSP polypeptide selected from the group consisting of: a polypeptide comprising amino acid residues from about from about Ser50 to about Leu66 of SEQ ID NO: 2; a polypeptide comprising amino acid residues from about Glu97 to about Leu105 of SEQ ID NO: 2; a polypeptide comprising amino acid residues from about Glu141 to about Gln148 of SEQ ID NO: 2; and a polypeptide comprising amino acid residues from about Asp219 to about Leu227 of SEQ ID NO: 2.

10. A method for making a recombinant vector comprising inserting an isolated nucleic acid molecule of claim 1 into a vector.

11. A recombinant vector produced by the method of claim 10.

12. A method of making a recombinant host cell comprising introducing the recombinant vector of claim 11 into a host cell.

13. A recombinant host cell produced by the method of claim 12.

14. A recombinant method for producing a hPSP polypeptide, comprising culturing the recombinant host cell of claim 13 under conditions such that said polypeptide is expressed and recovering said polypeptide.

15. An isolated hPSP polypeptide comprising an amino acid sequence at least 95% identical to a sequence selected from the group consisting of:
(a) the amino acid sequence from about position −18 to about position +231 in SEQ ID NO: 2 or the complete amino acid sequence encoded by the cDNA clone contained in ATCC Deposit No. 97811;
(b) the amino acid sequence from about position −17 to about position +231 in SEQ ID NO: 2 or the complete amino acid sequence except the N-terminal methionine encoded by the cDNA clone contained in ATCC Deposit No. 97811; and
(c) the amino acid sequence of the mature form of the hPSP polypeptide having the amino acid sequence from about position +1 to about position +231 in of SEQ ID NO: 2, or as encoded by the cDNA clone contained in the ATCC Deposit No.97811.

16. An isolated polypeptide comprising an epitope-bearing portion of the hPSP amino acid sequence, wherein said portion is selected from the group consisting of: a polypeptide comprising amino acid residues from about Ser5O to about Leu66 of SEQ ID NO: 2; a polypeptide comprising amino acid residues from about Glu97 to about Leu105 of SEQ ID NO: 2; a polypeptide comprising amino acid residues from about Glu141 to about Gln148 of SEQ ID NO: 2; and a polypeptide comprising amino acid residues from about Asp219 to about Leu227 of SEQ ID NO: 2.

17. An isolated antibody that binds specifically to an hPSP polypeptide of claim 15.

18. An isolated nucleic acid molecule comprising a polynucleotide having a sequence at least 95% identical to a sequence selected from the group consisting of
(a) a portion of the sequence shown in SEQ ID NO: 1, wherein said portion comprises at least 30 contiguous nucleotides from nucleotide 48 to nucleotide 793 but wherein said portion does not have the sequence of any one of SEQ ID NOS: 10-18 or any subfragment thereof; and
(b) a nucleotide sequence complementary to any of the nucleotide sequences in (a) above.
